(19) United States
(12) Patent Application Publication
(10) Pub. No.: US 2023/0338166 A1
(43) Pub. Date: Oct. 26, 2023

(54) GLENOSPHERE BASE REMOVAL TOOL

(71) Applicant: Shukla Medical, St. Petersburg, FL (US)

(72) Inventor: Nicholas Christopher Keach, Lutz, FL (US)

(73) Assignee: Shukla Medical, St. Petersburg, FL (US)

(21) Appl. No.: 17/724,719

(22) Filed: Apr. 20, 2022

(51) Int. Cl. A61F 2/46

(52) U.S. Cl. CPC A61F 2/4612; A61F 2002/4619; A61F 2002/4085

(57) ABSTRACT

A glenosphere base removal tool is provided. The tool includes a housing having an axis of rotation; a handle coupled to the housing for receiving a loosening torque from a user; a plurality of pin assemblies in the housing for engaging fastener holes of a glenosphere base, each of the pin assemblies extending substantially parallel to the axis of rotation and spaced radially from the axis of rotation by a radial distance; a cam assembly structured to adjust the radial distances of the pin assemblies; and a ratchet assembly structured to transfer the loosening torque to the pin assemblies for unscrewing the glenosphere base.

### BACKGROUND OF THE INVENTION

Reverse shoulder implants consist of various parts, including a round metallic glenosphere coupled to a glenosphere base. The base is typically formed integrally with a central anchoring screw and holes for receiving additional screw-like fasteners that secure the base to the scapula of a patient.

Reverse shoulder implants sometimes need removal or replacement due to complications that develop over time, such as damaging wear, infections, breakage etc. Removal of such an implant typically requires that the glenosphere base also be removed or replaced.

### SUMMARY OF THE INVENTION

In accordance with an exemplary embodiment of the subject disclosure, a glenosphere base removal tool is provided. The tool includes a housing having an axis of rotation; a handle coupled to the housing for receiving a loosening torque from a user; a plurality of pin assemblies in the housing for engaging fastener holes of a glenosphere base, each of the pin assemblies extending substantially parallel to the axis of rotation and spaced radially from the axis of rotation by a radial distance; a cam assembly structured to adjust the radial distances of the pin assemblies; and a ratchet assembly structured to transfer the loosening torque to the pin assemblies for unscrewing the glenosphere base.

In accordance with an aspect of the subject disclosure, the handle includes an outer surface provided with ergonomic features.

In accordance with another aspect of the subject disclosure, the ratchet assembly includes a gear wheel having teeth, a pawl and a biasing member to bias the pawl against the teeth.

In accordance with still another aspect of the subject disclosure, the biasing member is a spring.

In accordance with yet another aspect of the subject disclosure, the gear wheel further includes a floor having a plurality of radial slots, and the pin assemblies extend respectively though the radial slots.

In accordance with still another aspect of the subject disclosure, the pawl transfers the loosening torque to the gear wheel, and the gear wheel transfers the loosening torque to the pin assemblies.

In accordance with yet another aspect of the subject disclosure, the ratchet assembly does not transfer a tightening torque to the pin assemblies.

In accordance with still another aspect of the subject disclosure, the housing includes a through-bore and a pawl receptacle, the gear wheel is rotatably seated within the through-bore, and the pawl is positioned within the pawl receptacle.

In accordance with yet another aspect of the subject disclosure, the cam assembly includes a cam wheel having a plurality of spiral slots and a dial coupled to the cam wheel, and the pin assemblies extend respectively through the spiral slots.

In accordance with still another aspect of the subject disclosure, the biasing member is a spring.

In accordance with yet another aspect of the subject disclosure, the gear wheel includes a cam receptacle and the cam wheel is rotatably seated within the cam receptacle of the gear wheel.

In accordance with still another aspect of the subject disclosure, the through-bore has an axis coincident with the axis of rotation.

In accordance with yet another aspect of the subject disclosure, the plurality of pin assemblies consists of two pin assemblies.

In accordance with another exemplary embodiment of the subject disclosure, there is provided a glenosphere base removal tool for removing an installed glenosphere base having a plurality of fastener holes. The tool includes a housing having a main body, a top plate coupled to the main body, and an axis of rotation, the main body including a proximal end, a distal end having a through-bore extending approximately perpendicularly to the longitudinal axis, and a tapered and blind pawl receptacle communicating with the through-bore; a handle coupled to the proximal end of the housing; a ratchet assembly having a gear wheel rotatably positioned within the through-bore of the housing, a pawl positioned within the pawl receptacle of the housing, and a biasing member biasing pawl against the gear wheel, the gear wheel including a cam receptacle having a floor provided with two diametrically opposed radial slots; a cam assembly including a dial and a cam wheel coupled to the dial, the cam wheel rotatably positioned within the cam receptacle of the gear wheel and including a bottom surface provided with two concentric and opposed spiral slots; and two pin assemblies in the housing and extending substantially parallel to the axis of rotation, each of the pin assemblies extending through a respective radial slot of the gear wheel and a respective spiral slot of the cam wheel.

In accordance with an aspect of the subject disclosure, the housing has a longitudinal length of about 60 to 70 mm, including 65.7 mm and a height of about 12 to 13 mm, including 12.9 mm, and the through-bore of the housing has a diameter of about 15 to 29 mm, including 28 mm.

In accordance with another aspect of the subject disclosure, the handle includes a shaft having a longitudinal length of about 125 to 130 mm, including 127 mm.

### BRIEF DESCRIPTION OF THE SEVERAL VIEWS OF THE DRAWINGS

The following detailed description of an exemplary embodiment of the subject disclosure will be better understood when read in conjunction with the appended drawings. For the purpose of illustrating the present disclosure, there is shown in the drawings an exemplary embodiment. It should be understood, however, that the subject application is not limited to the precise arrangements and instrumentalities shown.

FIG. 1 is a top perspective view of a glenosphere base removal tool in accordance with an exemplary embodiment of the subject disclosure;

FIG. 2 is a bottom perspective view of the glenosphere base removal tool of FIG. 1;

FIG. 3 is an exploded view of the glenosphere base removal tool of FIG. 1

FIG. 4A is a perspective view of a glenosphere base installed within bone;

FIG. 4B is a perspective view of the glenosphere base of FIG. 4A partially unscrewed from bone;

FIG. 5A is a perspective view of a ratchet assembly of the glenosphere base removal tool of FIG. 1;

FIG. 5B is a perspective view of a pawl and biasing member of the ratchet assembly of FIG. 5A;

FIG. 6 is a top view of the ratchet assembly of FIG. 5A within a main housing body of the glenosphere base removal tool of FIG. 1;

FIG. 7A is a perspective view of a cam assembly of the glenosphere base removal tool of FIG. 1;

FIG. 7B is a top view of a cam wheel of the cam assembly of FIG. 7A;

FIG. 7C is a perspective view of the cam wheel of FIG. 7B;

FIG. 8A is a top view of assembled cam wheel, gear wheel and pin assemblies of the glenosphere base removal tool of FIG. 1, with the pin assemblies spaced apart from an axis of rotation;

FIG. 8B is a perspective view of the assembled cam wheel, gear wheel and pin assemblies of FIG. 8A;

FIG. 9A is a top view of the assembled cam wheel, gear wheel and pin assemblies of FIG. 8A, with the pin assemblies spaced close to the axis of rotation;

FIG. 9B is a perspective view of the assembled cam wheel, gear wheel and pin assemblies of FIG. 9A; and

FIG. 10 is a diagram detailing a process for using the glenosphere base removal tool of FIG. 1 to remove an installed glenosphere base.

### DETAILED DESCRIPTION OF THE INVENTION

Reference will now be made in detail to an exemplary embodiment of the subject disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as upper, lower, top, bottom, above, below and diagonal, are used with respect to the accompanying drawings. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or +0.1% from the specified value, as such variations are appropriate.

“Substantially” as used herein shall mean considerable in extent, largely but not wholly that which is specified, or an appropriate variation therefrom as is acceptable within the field of art.

“Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more exemplary embodiments. One skilled in the relevant art will recognize, in light of the description herein, that the subject disclosure can be practiced without one or more of the specific features or advantages of a particular exemplary embodiment. In other instances, additional features and advantages may be recognized in certain exemplary embodiments that may not be present in all exemplary embodiments of the present disclosure.

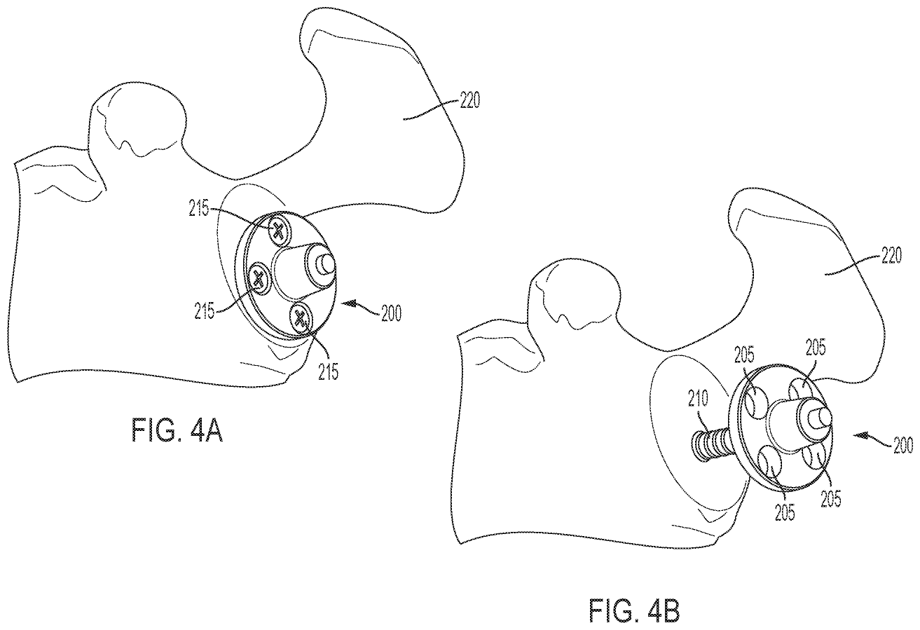

Referring now to the Figures, there is shown an exemplary glenosphere base removal tool 100 in accordance with the subject disclosure for loosening and/or removing an installed glenosphere base 200 of a glenosphere (not shown). As shown in FIGS. 4A and 4B, glenosphere base 200 includes a central anchoring screw 210 and four fastener holes 205 sized to receive glenosphere fasteners 215 for affixing base 200 to bone 220, such as a scapula, in a known manner.

Glenosphere base removal tool 100 includes a housing 500 having an axis of rotation 502, a handle 400 coupled to housing 500 for receiving a loosening torque from a user, a plurality of pin assemblies 600 in housing 500 for engaging fastener holes 205 of glenosphere base 200, a cam assembly 700 structured to adjust the radial positions of pin assemblies 600 to align with fastener holes 205, and a ratchet assembly 800 structured to transfer the loosening torque applied to handle 400 to pin assemblies 600 for unscrewing glenosphere base 200.

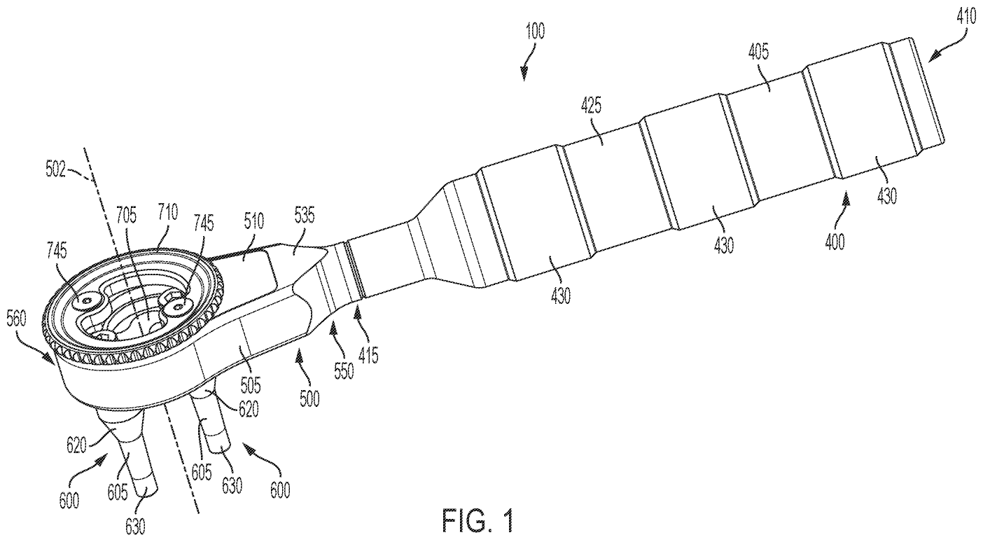

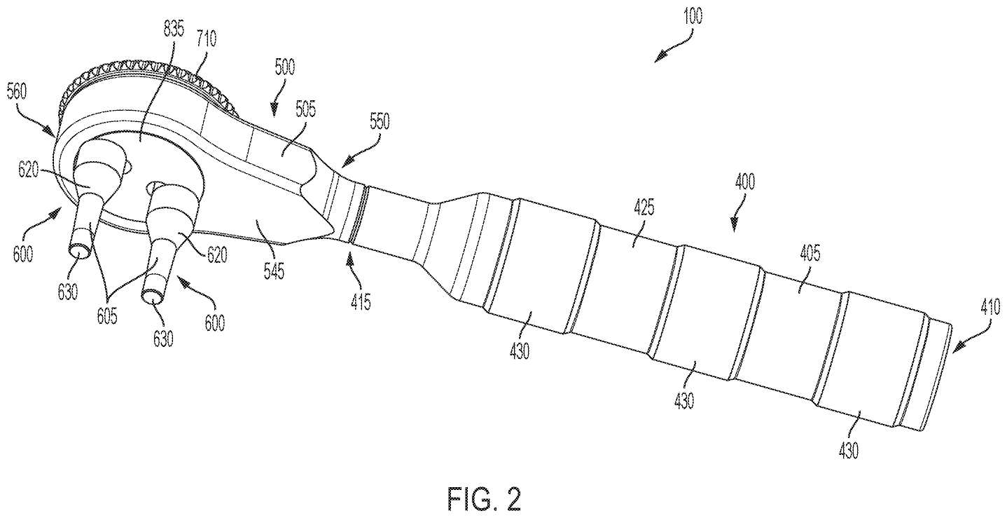

With reference to FIGS. 1-3, handle 400 includes a shaft 405 having a proximal end 410 for gripping by a user's hand, a tapered distal end 415 provided with screw threads 420 for coupling to housing 500, and an outer surface 425 provided with ergonomic features 430 to improve grip with the user's hand. With respect to at least some embodiments, shaft 405 has an overall longitudinal length of about 125 to 130 mm, including 127 mm, though it should be appreciated that shaft 405 may have a different length. Further, although ergonomic features 430 of handle 400 are illustrated in the drawings as raised rings circumscribing and formed integrally with shaft 405, it should be appreciated that ergonomic features 430 need not be formed integrally with shaft 405 and/or may include other features for improving a user's grip, such as, for example, texturing, stippling, a rubber coating, or the like. It should also be appreciated that shaft 405 need not include ergonomic features at all, such as, for example, with respect to embodiments incorporating a smooth shaft 405.

Housing 500 includes a main body 505 and a top plate 510 coupled to main body 505. Top plate 510 includes a top side 515, a bottom side 520 and a distal end 525 having a hole 530. Main body 505 is generally tear-shaped and includes a top end 535 having a shallow receptacle 540 sized to closely receive top plate 510, a bottom end 545, a proximal end 550 having a threaded hole 555 to receive screw threads 420 of handle 400 for rigidly coupling main body 505 to handle 400, a distal end 560 having a through-bore 565 axially aligned with hole 530 of top plate 510 and extending from top end 535 to bottom end 545 approximately perpendicularly to longitudinal axis 570, an internal annular ledge 575 about through-bore 565 at bottom end 545, and a tapered and blind pawl receptacle 580 communicating with through-bore 565 and closed at its top side by top plate 510. With respect to at least some embodiments, housing 500 has an overall longitudinal length of about 60 to 70 mm, including 65.7 mm, a top-to-bottom height of about 12 to 13 mm, including 12.9 mm, and/or through-bore 565 has a diameter of about 15 to 29 mm, including 28 mm, though it should be appreciated that these features may be dimensioned differently.

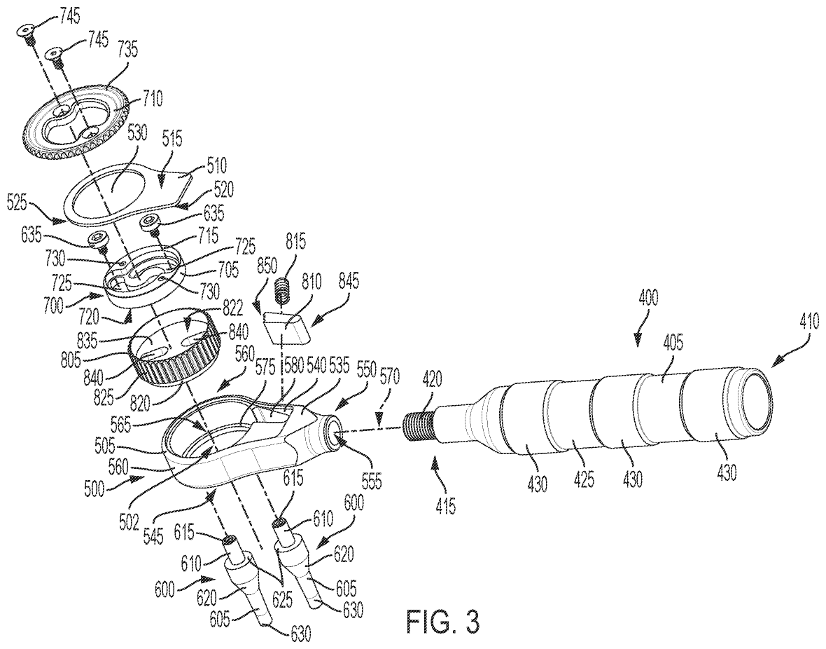

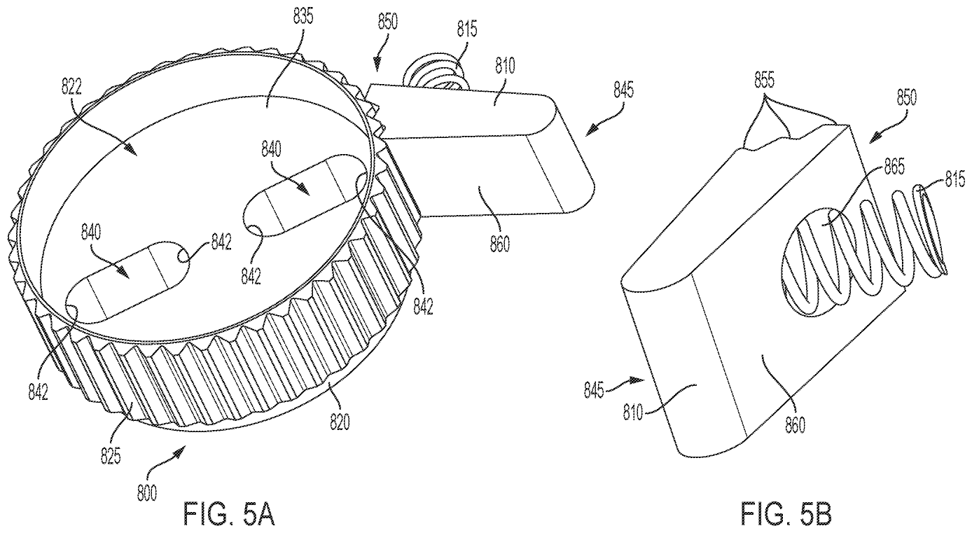

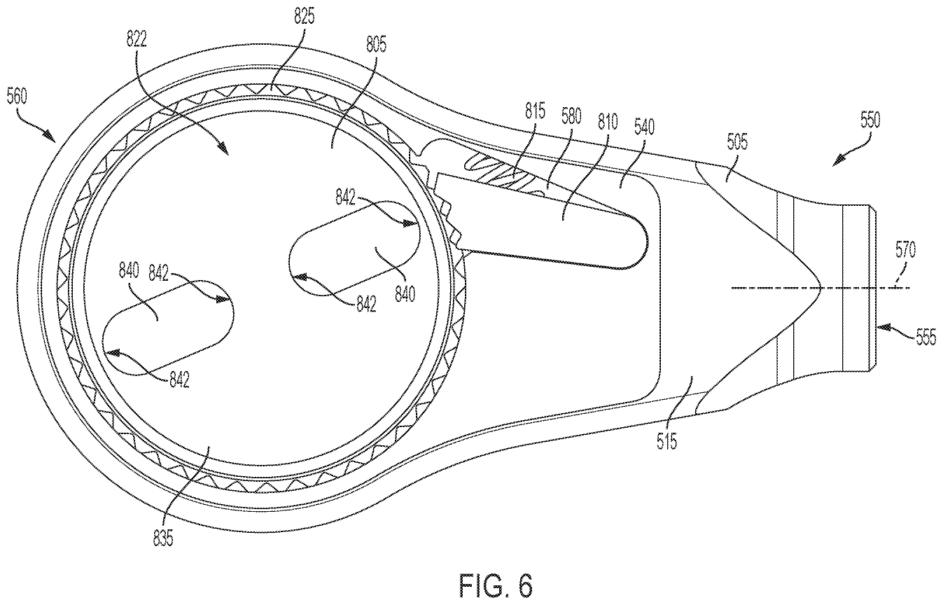

As best shown in FIGS. 3, 5A, 5B and 6, ratchet assembly 800 includes a gear wheel 805, a pawl 810, and a biasing member 815 to bias pawl 810 against gear wheel 805. Although biasing member 815 of the illustrated embodiments is a spring 815, it should be appreciated that biasing member 815 may include other structures in addition to or in lieu of spring 815 for biasing pawl 810, and that various embodiments described and claimed herein are not intended to be limited to any particular type of biasing member.

Gear wheel 805 of ratchet assembly 800 includes a cylindrical body 820 coaxial with through-bore 565 of housing 500, teeth 825 circumscribing a top end of cylindrical body 820, a floor 835 closing a lower end of cylindrical body 820 and forming a cam receptacle 822 at the top end of cylindrical body 820, and diametrically opposed radial slots 840 with ends 842 extending through floor 835. Gear wheel 805 is rotatably seated within through-bore 565 of housing 500 and constrained axially therein by top plate 510 and annular ledge 575 of main body 505.

Pawl 810 of ratchet assembly 800 includes a tapered and rounded proximal end 845, a distal end 850 provided with steps 855, and an outer surface 860 having an angled receptacle 865 for receiving biasing member 815. Pawl 810 is positioned within pawl receptacle 580 and pivotable therein about proximal end 845. Biasing member 815 is positioned within receptacle 865 to bias steps 855 of pawl 810 against teeth 825 of gear wheel 805. In this manner, pawl 810 provides a one-way ratcheting function by allowing counterclockwise rotation of gear wheel 805 relative to housing 500 (when viewed from the top of glenosphere base removal tool 100), while preventing clockwise rotation thereof.

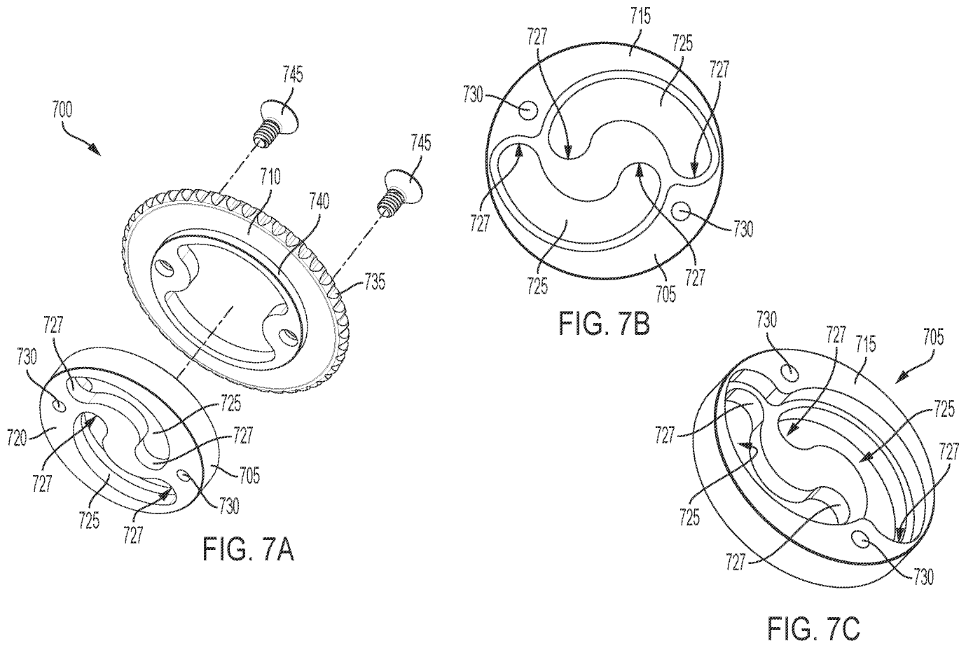

As best shown in FIGS. 3 and 7A-7C, cam assembly 700 includes a cam wheel 705 and a user-adjustable dial 710 coupled to cam wheel 705. Cam wheel 705 is rotatably positioned within cam receptacle 822 of gear wheel 805 and includes a top surface 715 abutting top plate 510 of housing 500, a bottom surface 720 abutting floor 835 of gear wheel 805, two concentric and opposed spiral slots 725 with ends 727 extending top-to-bottom therethrough, and fastener holes 730. Dial 710 includes a grip 735 and a cylindrical lower extension 740 seated rotatably within hole 530 of top plate 510 and coupled to top surface 715 of cam wheel 705 via fasteners 745. Although screw-like fasteners 745 are shown in the drawings, it should be appreciated that fasteners 745 may include other structures for fastening dial 710 to cam wheel 705, such as, for example, bolts, pins, glue, and the like, and that various embodiments described and claimed herein are not intended to be limited to any particular type(s) of fasteners.

Each pin assembly 600 includes a pin 605 having a cylindrically shaped top end 610 with a fastener hole 615, a tapered mid-portion 620 having an annular engagement face 625, and a slightly tapered and approximately cylindrically shaped lower end 630 sized to engage a fastener hole 205 of glenosphere base 200. Top end 610 of each pin 605 extends slidingly and upwardly through a respective one of radial slots 840 of gear wheel 805 and a respective one of spiral slots 725 of cam wheel 705. Fasteners 635 engage with fastener holes 615 to maintain pins 605 within and prevent their removal from housing 500, while annular engagement faces 625 of pins 605 engage with the bottom face of floor 835 of gear wheel 805 to maintain pins 605 in an approximately perpendicular orientation relative to housing 500 and parallel to axis of rotation 502.

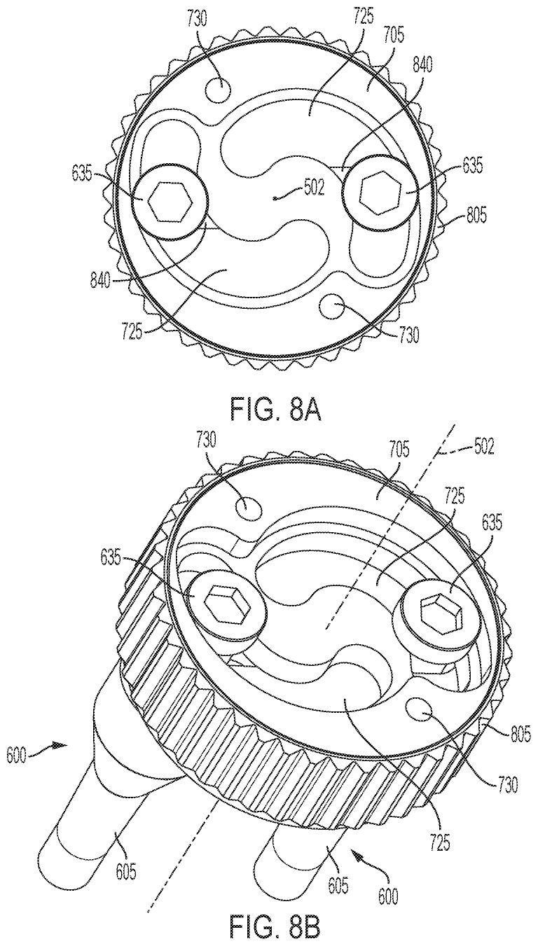

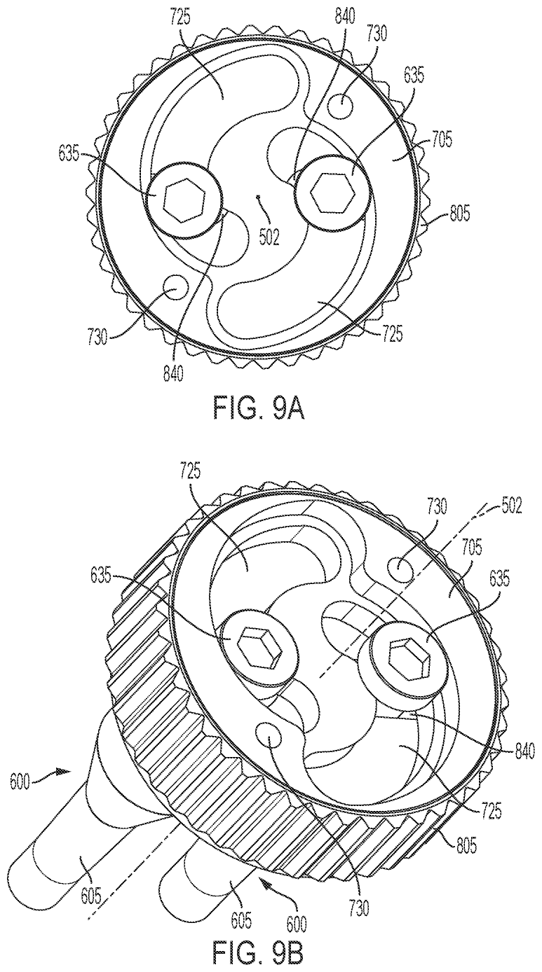

Cam assembly 700 is operable to permit adjustment of the orientation of pin assemblies 600 to align with fastener holes of various different sizes and brands of glenosphere bases, such as, for example, fastener holes 205 of glenosphere base 200. To make the adjustment, a user (e.g., a surgeon, technician or the like) rotates grip 735 of dial 710, which in turn causes cam wheel 705 to rotate within gear wheel 805. Depending on the direction of rotation of dial 710, rotation of cam wheel 705 causes the sides of spiral slots 725 to urge pin assemblies 600 radially within respective radial slots 840 of gear wheel 805 either toward axis of rotation 502 (see FIGS. 9A and 9B) or away from axis of rotation 502 (see FIGS. 8A and 8B). The range of rotation of dial 705 in either direction is limited by ends 727, 842 of spiral and radial slots 725, 840, respectively, which in turn limit the range of radial positions of pin assemblies 600. Diametric symmetry of spiral slots 725 and radial slots 840 also ensures that pin assemblies 600 remain equidistant from axis of rotation 502 at all radial positions.

With respect to the embodiments illustrated in the Figures, clockwise rotation of dial 710 (when viewed from the top of glenosphere base removal tool 100) urges pin assemblies 600 radially toward axis of rotation 502, and vice versa for counterclockwise rotation, though it should be appreciated that counterclockwise rotation of dial 710 may urge pin assemblies 600 toward axis of rotation 502 in other embodiments, and that various embodiments described and claimed herein are not intended to be limited to any particular rotational convention. Further, while cam assembly 700 and ratchet assembly 800 are structured to accommodate two pin assemblies 600, it should be appreciated that cam assembly 700 and ratchet assembly 800 may be provided with additional slots 725, 840 to accommodate more pin assemblies 600, and that various embodiments described and claimed herein are not intended to be limited to any specific number of pin assemblies 600.

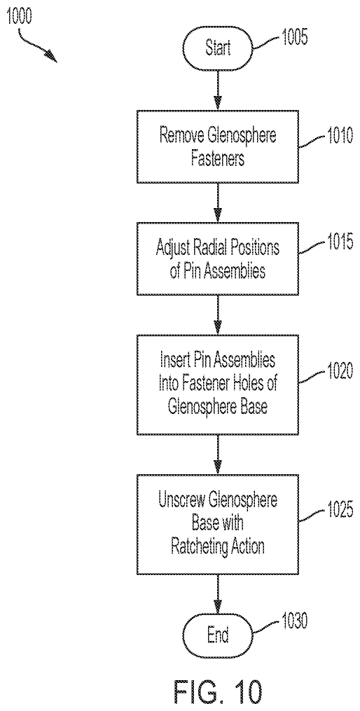

Referring now to FIG. 10, there is shown a diagram detailing a process 1000 for using glenosphere base removal tool 100 to remove an installed glenosphere base 200. The process begins at step 1005 and proceeds to step 1010, at which a user removes glenosphere fasteners 215 from fastener holes 205 of glenosphere base 200 using an appropriate tool, such as a screwdriving tool (see FIGS. 4A and 4B). Then, at step 1015, the user operates grip 735 of dial 710 to adjust the radial positions of pin assemblies 600 to align with two opposed fastener holes 205 of glenosphere base 200. Once pin assemblies 600 are aligned, the process proceeds to step 1020, at which the user inserts lower ends 630 of pins 605 into the opposed fastener holes 205. Then, at step 1025, the user operates handle 400 in a ratcheting action to unscrew glenosphere base 200 from bone 220. Counterclockwise rotation of the ratcheting action (as viewed from the top of glenosphere base removal tool 100) causes steps 855 of pawl 810 to engage and lock with teeth 825 of gear wheel 805 which, in turn, transfers a counterclockwise loosening torque to gear wheel 805. This counterclockwise loosening torque is applied to pin assemblies 600 via the sides of radial slots 840 of gear wheel 805, thereby causing glenosphere base 200 to unscrew from bone 220 in counterclockwise fashion. Clockwise rotation of the ratcheting action causes pawl 810 to “click” and slide along the circumference of gear wheel 805 to prevent a clockwise tightening torque from being applied to gear wheel 805. In this manner, glenosphere base removal tool 100 advantageously prevents damage that may occur from inadvertent tightening of glenosphere base 200, such as, for example, stripping of anchoring screw 210 of base 200, cracking of bone 220, etc. Once glenosphere base 200 is unscrewed and removed, the process ends at step 1030.

It should be appreciated by those skilled in the art that changes may be made to the exemplary embodiments described above without departing from the broad inventive concept thereof. It is to be understood, therefore, that this disclosure is not limited to the particular exemplary embodiments disclosed, but it is intended to cover modifications within the spirit and scope of the claims defined herein.

What is claimed is:

1. A glenosphere base removal tool for removing an installed glenosphere base having a plurality of fastener holes, the tool comprising:
a housing having an axis of rotation;
a handle coupled to the housing for receiving a loosening torque from a user;
a plurality of pin assemblies in the housing for engaging the fastener holes of the glenosphere base, each of the pin assemblies extending substantially parallel to the axis of rotation and spaced radially from the axis of rotation by a radial distance;
a cam assembly structured to adjust the radial distances of the pin assemblies; and
a ratchet assembly structured to transfer the loosening torque to the pin assemblies for unscrewing the glenosphere base.

2. The glenosphere base removal tool of claim 1, wherein the handle includes an outer surface provided with ergonomic features.

3. The glenosphere base removal tool of claim 1, wherein the ratchet assembly includes a gear wheel having teeth, a pawl and a biasing member to bias the pawl against the teeth.

4. The glenosphere base removal tool of claim 3, wherein the biasing member is a spring.

5. The glenosphere base removal tool of claim 3, wherein the gear wheel further includes a floor having a plurality of radial slots, and the pin assemblies extend respectively though the radial slots.

6. The glenosphere base removal tool of claim 5, wherein the pawl transfers the loosening torque to the gear wheel, and the gear wheel transfers the loosening torque to the pin assemblies.

7. The glenosphere base removal tool of claim 6, wherein the ratchet assembly does not transfer a tightening torque to the pin assemblies.

8. The glenosphere base removal tool of claim 3, wherein the housing includes a through-bore and a pawl receptacle, the gear wheel is rotatably seated within the through-bore, and the pawl is positioned within the pawl receptacle.

9. The glenosphere base removal tool of claim 1, wherein the cam assembly includes a cam wheel having a plurality of spiral slots and a dial coupled to the cam wheel, and the pin assemblies extend respectively through the spiral slots.

10. The glenosphere base removal tool of claim 9, wherein the housing includes a through-bore and a pawl receptacle, the ratchet assembly includes a gear wheel with teeth rotatably seated within the through-bore, a pawl within the pawl receptacle and a biasing member to bias the pawl against the teeth.

11. The glenosphere base removal tool of claim 10, wherein the biasing member is a spring.

12. The glenosphere base removal tool of claim 10, wherein the gear wheel includes a cam receptacle and the cam wheel is rotatably seated within the cam receptacle of the gear wheel.

13. The glenosphere base removal tool of claim 12, wherein the gear wheel further includes a floor having a plurality of radial slots, and the pin assemblies extend respectively though the radial slots.

14. The glenosphere base removal tool of claim 13, wherein the through-bore has an axis coincident with the axis of rotation.

15. The glenosphere base removal tool of claim 1, wherein the plurality of pin assemblies consists of two pin assemblies.

16. A glenosphere base removal tool for removing an installed glenosphere base having a plurality of fastener holes, the tool comprising:
a housing having a main body, a top plate coupled to the main body, and an axis of rotation, the main body including a proximal end, a distal end having a through-bore extending approximately perpendicularly to the longitudinal axis, and a tapered and blind pawl receptacle communicating with the through-bore;
a handle coupled to the proximal end of the housing;
a ratchet assembly having a gear wheel rotatably positioned within the through-bore of the housing, a pawl positioned within the pawl receptacle of the housing, and a biasing member biasing pawl against the gear wheel, the gear wheel including a cam receptacle having a floor provided with two diametrically opposed radial slots;
a cam assembly including a dial and a cam wheel coupled to the dial, the cam wheel rotatably positioned within the cam receptacle of the gear wheel and including a bottom surface provided with two concentric and opposed spiral slots; and
two pin assemblies in the housing and extending substantially parallel to the axis of rotation, each of the pin assemblies extending through a respective radial slot of the gear wheel and a respective spiral slot of the cam wheel.

17. The glenosphere base removal tool of claim 16, wherein the housing has a longitudinal length of about 60 to 70 mm, including 65.7 mm and a height of about 12 to 13 mm, including 12.9 mm, and the through-bore of the housing has a diameter of about 15 to 29 mm, including 28 mm.

18. The glenosphere base removal tool of claim 16, wherein the handle includes a shaft having a longitudinal length of about 125 to 130 mm, including 127 mm.

19. The glenosphere base removal tool of claim 16, wherein the handle includes an outer surface provided with ergonomic features.
